(19) United States
(12) Patent Application Publication
(10) Pub. No.: US 2012/0252133 A1
(43) Pub. Date: Oct. 4, 2012

(54) TEST METER WITH A STRIP PORT CONNECTOR CONFIGURED FOR FLUID ENTRAPMENT

(75) Inventors: Allan FAULKNER, Avoch (GB); Jonathan Nelson, Inverness (GB); Nicholas Foley, Edinburgh (GB); Simon Salter, Edinburgh (GB); David Colin Crosland, Edinburgh (GB); Paul Trickett, Hamilton (GB); Gavin Flowers, Edinburgh (GB); Martin Crofton, London (GB)

(73) Assignee: Cilag GmbH International, Zug (CH)

(21) Appl. No.: 13/077,786

(22) Filed: Mar. 31, 2011

(51) Int. Cl. G01N 1/00; C12M 1/34; G01N 33/48

(52) U.S. Cl. 436/174; 422/68.1; 435/287.1

(57) ABSTRACT

A test meter for use win an analytical test strip for the determination of an analyte (such as glucose) in a bodily fluid sample (e.g., a whole blood sample) includes a test meter housing and a strip port connector. The test meter housing has a distal end with a strip port opening configured to receive the analytical test strip. The strip port connector is integrated with the distal end of the test meter housing and has a distal portion with an analytical test strip guide. The analytical test strip guide has an upper guide surface configured such that an analytical test strip received in the strip port opening is guided and supported by the upper guide surface and also has a liquid entrapment feature configured to direct free liquid that enters the strip port opening away from the upper guide surface and entrap such directed liquid in the distal portion of the strip port connector and/or the distal end of the test meter housing. The strip port connector also has a proximal portion disposed within the test meter housing with the proximal portion having an electrical connector pin configured for operable electrical contact with an analytical test strip received within the strip port opening.

### BACKGROUND OF THE INVENTION

1. Field of the Invention

The present invention relates, in general, to medical devices and, in particular, to test meters for use with analytical test strips and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using analytical test strips along with an associated test meter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified perspective view of a test meter (shown with an analytical test strip received therein) according to an embodiment of the present invention;

FIG. 2 is a simplified perspective angled view of a portion of the test meter of FIG. 1;

FIG. 3 is a simplified perspective end view of a portion of the test meter of FIG. 1;

FIG. 4 is a simplified perspective view of a portion of a strip port connector integrated into the test meter of FIG. 1;

FIG. 5 is a simplified cross-sectional side view of a portion of the test meter of FIG. 1 with an analytical test strip received therein;

FIGS. 6A, 6B, and 6C are simplified top, cross-sectional side (along line A-A of FIG. 6A) and front views of the analytical test strip guide of the test meter of FIG. 1;

FIG. 7 is a simplified top view of an analytical test strip guide as can be employed in test meters according to embodiments of the present invention; and

FIG. 8 is a flow diagram depicting stages in a method for employing a test meter for use with an analytical test strip in the determination of an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, test meters for use with an analytical test strip for the determination of an analyte (such as glucose) in a bodily fluid sample (e.g., a whole blood sample) according to the present invention include a test meter housing and a strip port connector, with the test meter housing having a housing outer surface and a distal end. Moreover, the housing outer surface has a housing distal surface with a strip port opening. The strip port opening, which extends through the housing distal surface, is configured to receive an analytical test strip. The strip port connector is integrated with the distal end of the test meter housing and has a distal portion. The strip port connector also includes an analytical test strip guide.

The analytical test strip guide of the strip port connector has at least one upper guide surface configured such that an analytical test strip received in the strip port opening is guided and supported by the upper guide surface and also has at least one liquid entrapment feature configured to direct free liquid that inadvertently enters the strip port connector opening away from the upper guide surface and entrap such directed liquid in the distal portion of the strip port connector and/or in the distal end of the test meter housing. The strip port connector also has a proximal portion disposed within the test meter housing, with the proximal portion having an electrical connector pin configured for operable electrical contact with an analytical test strip received within the strip port opening.

Test meters according to the present invention are beneficial in that free liquid (i.e., loose or unbound liquid) that inadvertently enters the test meter through the strip port opening is directed away from the upper guide surface and entrapped in the distal portion of the strip port connector and/or the distal end of the test meter housing. Such entrapped liquid is, therefore, prevented from entering the proximal portion of the strip port connector or the remainder of the test meter and deleteriously interfering with test meter operation by, for example, causing an electrical short between electrical connector pins. In this regard, it is noted that test meters are typically cleaned in a clinical setting or elsewhere using cleaning fluid applied via a cleaning fluid dampened or wetted cloth. During such cleaning, cleaning fluid that is separated from the cloth by, for example, being squeezed out of the cloth, can inadvertently enter the strip port opening and, if not prevented from doing so, travel to the proximal portion causing electrical problems and associated test meter errors. Such “free” cleaning fluid is an example of the “free liquid” that is directed away from the upper guide surface and entrapped in the distal portion of the strip port connector and/or distal end of the test meter housing in test meters according to embodiments of the present invention.

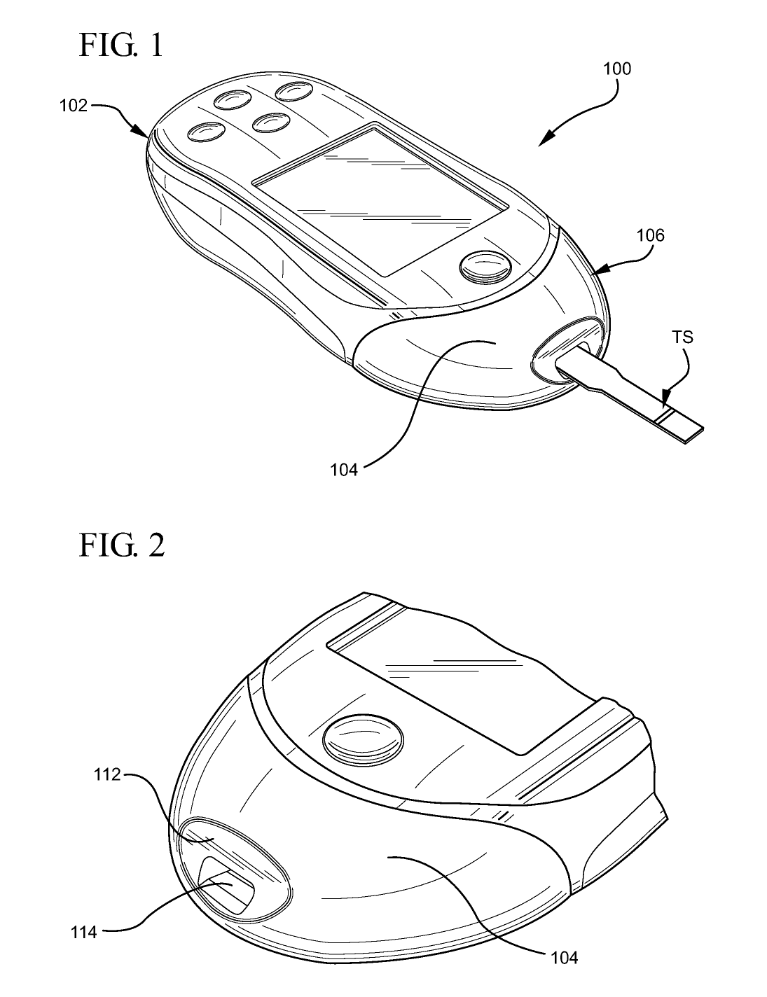

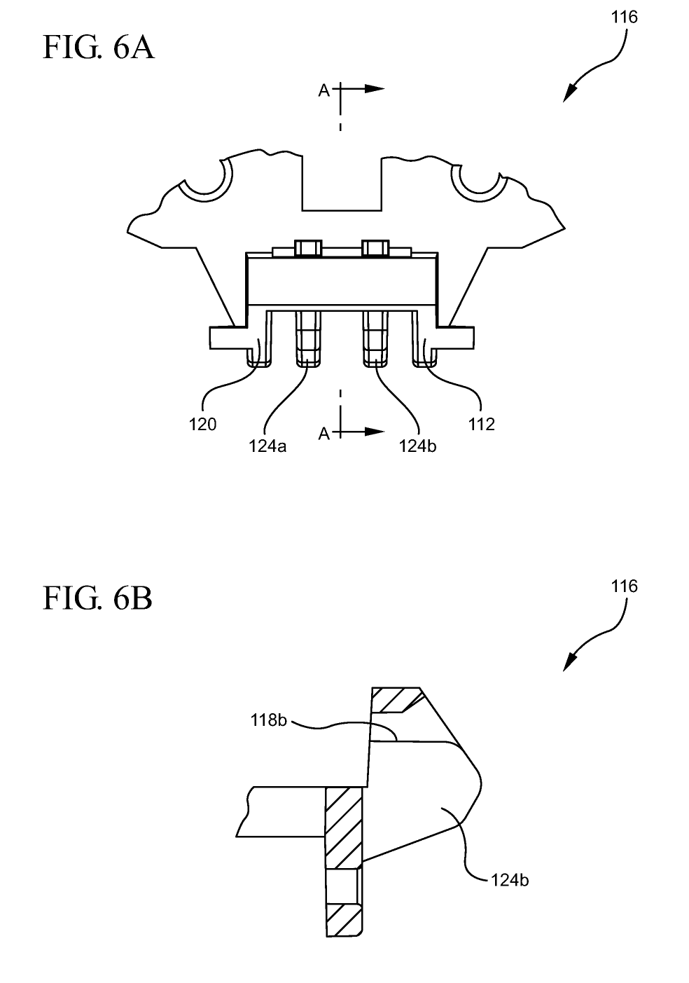

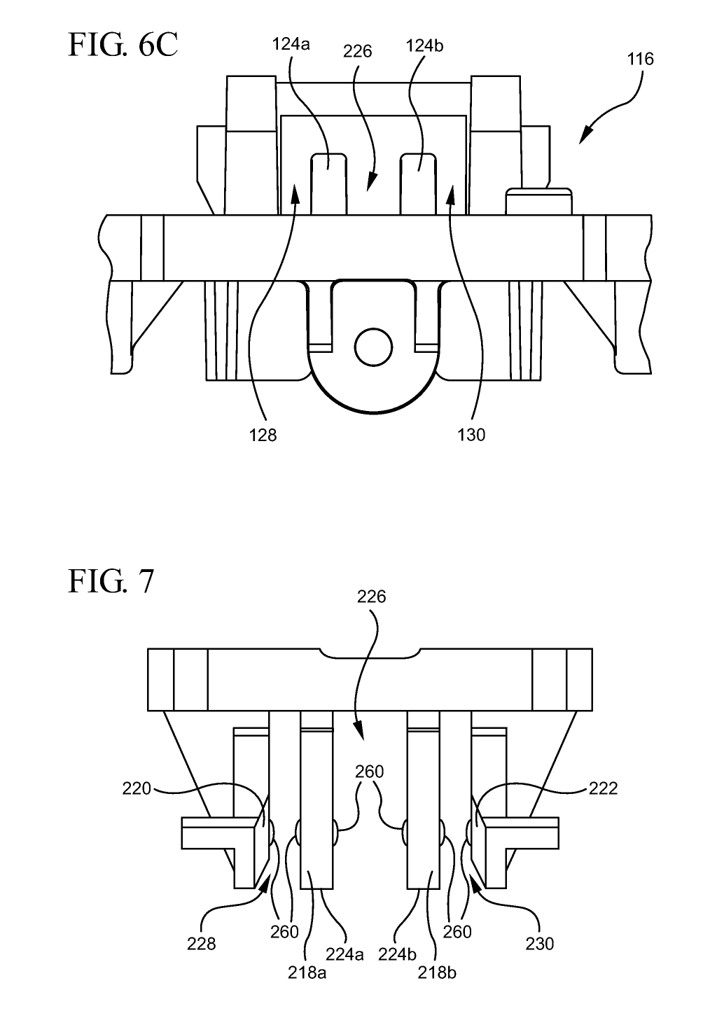

Referring to FIGS. 1-6C, test meter 100 for the determination of an analyte in a bodily fluid sample includes a test meter housing 102, with a housing outer surface 104 and a distal end 106, and a strip port connector 108 that is integrated within distal end 106 of test meter housing 102.

Test meter housing 102 can be formed of any suitable material including, for example, polycarbonate materials, thermo-plastic elastomer materials, acrylonitrile butadiene styrene (ABS) materials, and combinations thereof.

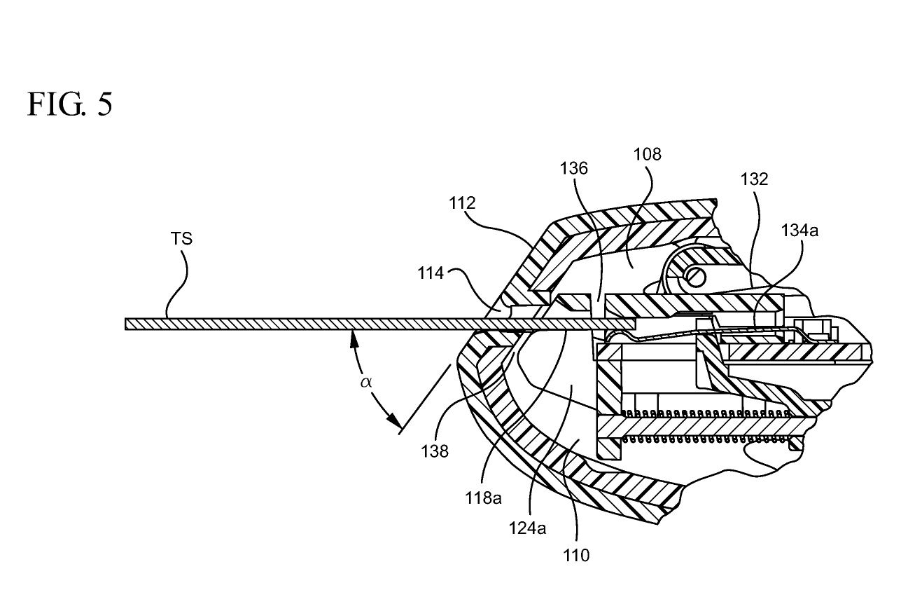

Strip port connector 108 includes a distal portion 110 and housing outer surface 104 of test meter housing 102 has a housing distal surface 112. In addition, housing distal surface 112 has a strip port opening 114 therethrough that is configured to receive analytical test strip TS (see FIGS. 1 and 5 in particular).

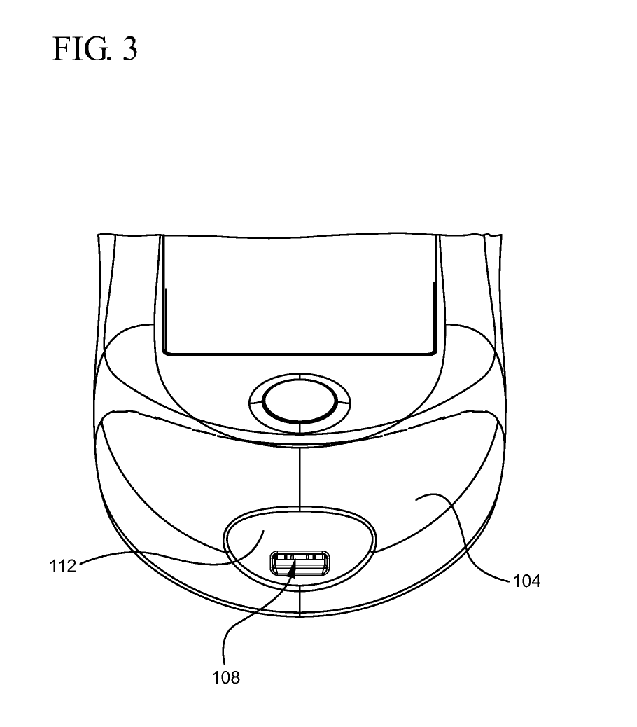

Strip port connector 108 also includes an analytical test strip guide 116 with two upper guide surfaces 118a and 118b configured such that analytical test strip TS received in strip port opening 114 is guided and supported by upper guide surfaces 118a and 118b. Analytical test strip guide 116 also includes sidewalls 120 and 122 configured to guide analytical test strip TS as it is received and removed from strip port opening 114. Sidewalls 120 and 122 also prevent inadvertent and potentially deleterious side-to-side movement (left-to-right and right-to-left in the perspective of FIG. 3) of an analytical test strip within strip port connector 108 during use of test meter 100.

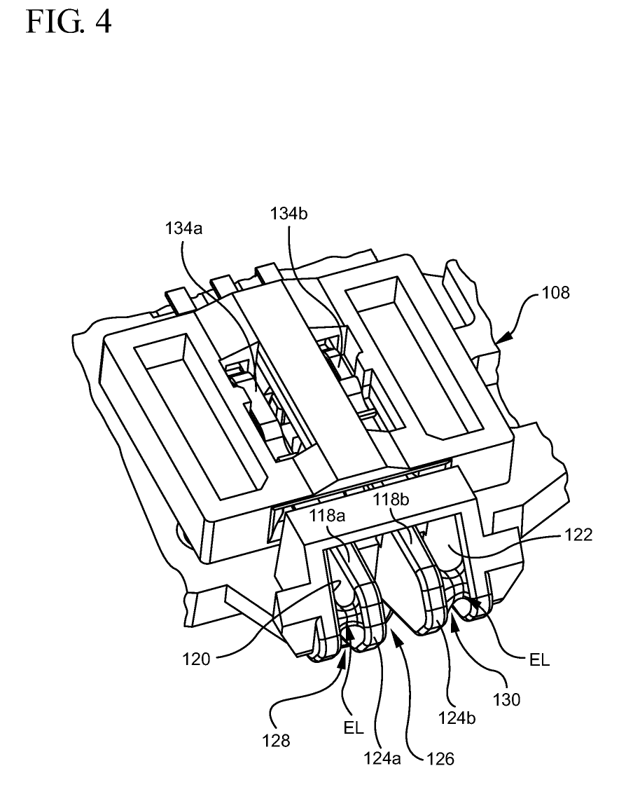

In the embodiment of FIGS. 1-6C, upper guide surfaces 118a and 118b are upper surfaces of guide rails 124a and 124b (also referred to as “ribs” 124a and 124b), respectively. Moreover, guide rails 124a and 124b are separated from one another by gap 126 and separated from sidewalls 120 and 122 by gaps 128 and 130 (see FIG. 4 in particular).

Gap 126, gap 128 and gap 130 are liquid entrapment features configured to direct free (loose, unbound) liquid that inadvertently enters strip port connector 108 opening away from upper guide surfaces 118a and 118b and entrap (i.e., restrain, keep) such directed liquid in distal portion 110 of strip port connector 108 and/or in the distal end 106 of test meter housing 102. FIG. 4 depicts entrapped liquid (EL) in gap 128 between rib 124a and sidewall 120 and entrapped liquid (EL) between rib 124b and sidewall 122 in gap 130.

To facilitate such direction and entrapment, ribs 124a and 124b can be formed of any suitable material including, for example, ABS materials, polycarbonate materials, hydrophilic materials, thermoplastic polymeric materials, glass-reinforced thermoplastic polymeric materials, and combinations thereof. Moreover, the widths of all or any of gap 126, gap 128 and gap 130 can be predetermined to provide for capillary forces that direct (i.e., draw) free liquid into the gap(s) and away from upper guide surfaces 118a and 118b. For example, gap 126 can have a width in the range of 0.25 mm to 2.00 mm, while gaps 128 and 130 can also have a width in the range of 0.25 mm to 2.00 mm.

Strip port connector 108 also includes a proximal portion 132 disposed within test meter housing 102. Proximal portion 132 includes electrical connector 134a and electrical connector 134b. Electrical connectors 134a and 134b are configured for operable electrical contact with an analytical test strip received within the strip port opening (see, for example, FIG. 5). To facilitate the direction and entrapment of liquid in the distal portion of the strip port connector, distal portion 110 is separated from proximal portion by gap 136 (see FIG. 5) and separated from test meter housing 102 by gap 138 (see FIG. 5).

Referring in particular to FIG. 5, in test meter 100, housing distal surface 112 is a flat outer surface. Moreover, this flat housing distal surface disposed at an acute angle alpha (α) with respect to analytical test strip TS received in the strip port connector opening. Acute angle α provides test meter 100 with a configuration wherein strip port opening 114 has an accessible (open) geometry. In other words, strip port opening 114 is accessible from the top of test meter 100 (in the orientation of FIG. 5) since the most distal portion of test strip opening 114 is only enclosed on three sides (see FIGS. 2 and 4 in particular). Such an accessible geometry beneficially enables a user to clean the test meter and the strip port opening without forcing a cloth dampened with cleaning liquid into a tight opening that is enclosed on four sides and thereby squeezing liquid from the cloth and creating free liquid in the strip port opening. Angle alpha (α) can be, for example, in the range of 25 degrees to less than 90 degrees.

FIG. 7 is a simplified top view of an analytical test strip guide 208 as can be employed in test meters according to embodiments of the present invention. Analytical test strip guide 208 includes two upper guide surfaces 218a and 218b that are configured to guide and support an analytical test strip. Analytical test strip guide 208 also includes sidewalls 220 and 222 configured to guide analytical test strip TS and six hemispherical protrusions 260.

In the embodiment of FIG. 7, upper guide surfaces 218a and 218b are upper surfaces of guide rails 224a and 224b, respectively. Moreover, guide rails 224a and 224b are separated from one another by gap 226 and separated from sidewalls 220 and 222 by gaps 228 and 230, respectively.

Gap 226, gap 228 and gap 230, as well as hemispherical protrusions 260, are liquid entrapment features configured to direct free (loose, unbound) liquid that inadvertently enters an associated strip port connector opening (not shown in FIG. 7) away from upper guide surfaces 218a and 218b and entrap (i.e., restrain, keep) such directed liquid in a distal portion of an associated strip port connector (also not shown in FIG. 7) and/or in a distal end of an associated test meter housing (not shown in FIG. 7).

Although for the purpose of illustration only, two guide rails and six hemispherical protrusions are depicted in FIG. 7, any suitable number of guide rails and protrusions and any suitably shaped protrusions can be employed.

In general, methods for employing a test meter for use with an analytical test strip in the determination of an analyte (such as glucose) in a bodily fluid sample (for example, a whole blood sample) according to the present invention include cleaning a test meter that includes a strip port opening with a liquid. In such a cleaning step, the test meter has a strip port connector that is integrated into a distal end of a test meter housing of the test meter and that includes an analytical test strip guide with a liquid entrapment feature(s). Moreover, the liquid entrapment feature(s) is configured to (i) direct free liquid that enters the strip port opening during cleaning away from an upper guide surface of the analytical test strip guide using, for example, capillary forces, gravitational forces or a combination thereof, and (ii) entrap such directed liquid in a distal portion of the strip port connector and/or the distal end of the test meter housing. The method also includes employing the test meter to determine an analyte in a bodily fluid sample using an analytical test strip received in the strip port connector.

Methods according to embodiments of the present invention are beneficial in that they enable cleaning of a test meter prior to its use in the determination of an analyte while also minimizing routes by which fluid used in the cleaning can be transferred into portions of the strip port connector where the fluid may cause harm. For example, during cleaning of a strip port connector using a cloth dampened with cleaning fluid, free cleaning fluid (i.e., cleaning fluid that has become separated from the cloth) can inadvertently enter the strip port connector via the strip port opening. However, in methods according to embodiments of the present invention, such free liquid is directed away from the upper guide surface and safely entrapped in the distal portion of the test strip connector and/or the distal portion of the test meter's housing.

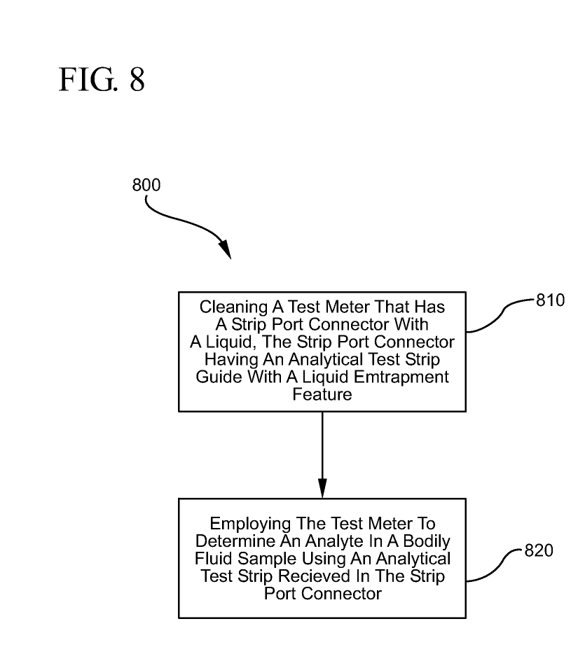

FIG. 8 is a flow diagram depicting stages in a method 800 for employing a test meter for use with an analytical test strip in the determination of an analyte (such as glucose) in a bodily fluid sample (for example, a whole blood sample). At step 810 of method 800, a test meter with a strip port opening is cleaned with a liquid. In this cleaning step, the test meter has a strip port connector integrated into a distal end of a test meter housing of the test meter. In addition, the strip port connector includes an analytical test strip guide with at least one liquid entrapment feature configured to direct free liquid that enters the strip port opening during cleaning away from at least one upper guide surface of the analytical test strip guide and to entrap such directed liquid in at least one of a distal portion of the strip port connector and the distal end of the test meter housing.

Step 810 can be accomplished, for example, using a cleansing liquid and a cloth that has been dampened or wetted with the cleansing liquid. Typical cleansing liquids include, but are not limited to, a 10% solution of sodium hypochlorite in water, hydrogen peroxide solutions, chlorohexidine solutions, 70% isopropyl alcohol and ethyl alcohol.

Method 800 also includes employing the test meter to determine an analyte in a bodily fluid sample using an analytical test strip received in the strip port connector (see step 820 of FIG. 8).

Once apprised of the present disclosure, one skilled in the art will recognize that methods according to embodiments of the present invention including method 800 can be readily modified to incorporate any of the techniques, benefits and characteristics of test meters according to embodiments of the present invention and described herein. For example, in method 800, the strip port connector can include (i) a distal portion with an outer surface contiguous with the housing outer surface, the outer surface having a strip port opening configured to receive the analytical test strip, and (ii) a proximal portion disposed within the test meter housing, the proximal portion having at least one electrical connector pin configured for operable electrical contact with an analytical test strip received within the strip port opening. Moreover, the upper guide surface of the analytical test strip guide can be configured such that the analytical test strip received in the strip port opening is guided and supported by the at least one upper guide surface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A test meter for use with an analytical test strip for the determination of an analyte in a bodily fluid sample, the test meter comprising:
a test meter housing with a housing outer surface and a distal end, the housing outer surface having a housing distal surface with a strip port opening therethrough, the strip port opening configured to receive an analytical test strip; and
a strip port connector integrated into the distal end of the test meter housing, the strip port connector including:
a distal portion with an analytical test strip guide that includes:
at least one upper guide surface configured such that an analytical test strip received in the strip port opening is guided and supported by the at least one upper guide surface; and
at least one liquid entrapment feature configured to direct free liquid that enters the strip port connector opening away from the upper guide surface and to entrap such directed liquid in at least one of the distal portion of the strip port connector and the distal end of the test meter housing; and
a proximal portion disposed within the test meter housing, the proximal portion having at least one electrical connector pin configured for operable electrical contact with an analytical test strip received within the strip port opening and supported by the at least one upper guide surface.

2. The test meter of claim 1 wherein the analytical test strip guide includes at least one guide rail with the at least one upper guide surface, and
wherein the at least one liquid entrapment feature is a gap adjacent the guide rail, the gap configured to direct free liquid that enters the strip port opening away from the upper guide surface of the guide rail and to entrap such directed liquid in at least one of the distal portion of the strip port connector and the distal end of the test meter housing

3. The test meter of claim 2 wherein the analytical test strip guide further includes at least one hemispherical protrusion configured to facilitate the directing of the free liquid.

4. The test meter of claim 4 wherein the at least one hemispherical protrusion is disposed on the at least one guide rail.

5. The test meter of claim 2 wherein the analytical test strip guide further includes at least one sidewall.

6. The test meter of claim 5 wherein the gap is between the guide rail and the sidewall.

7. The test meter of claim 1 wherein the housing outer surface is a flat outer surface.

8. The test meter of claim 7 wherein the housing outer surface is a flat angled housing outer surface disposed at an acute angle with respect to an analytical test strip received in the strip port opening.

9. The test meter of claim 8 wherein the combination of the flat angled housing outer surface and the strip port opening defines a strip port opening with an open geometry.

10. The test meter of claim 1 wherein the liquid entrapment feature is configured to direct free liquid that enters the strip port opening away from the upper guide surface using at least capillary forces.

11. The test meter of claim 1 wherein the liquid entrapment feature is configured to direct free liquid that enters the strip port connector opening away from the upper guide surface using at least gravitational forces.

12. The test meter of claim 1 wherein the test meter and analytical test strip are configured for the determination of glucose in a whole blood sample.

13. The test meter of claim 12 wherein the analytical test strip is an electrochemical-based analytical test strip.

14. A method for employing a test meter for use with an analytical test strip in the determination of an analyte in a bodily fluid sample, the method comprising:
cleaning a test meter that includes a strip port opening with a liquid, wherein the test meter has a strip port connector integrated into a distal end of a test meter housing of the test meter and the strip port connector includes an analytical test strip guide with at least one liquid entrapment feature configured to direct free liquid that enters the strip port opening during cleaning away from at least one upper guide surface of the analytical test strip guide and to entrap such directed liquid in at least one of a distal portion of the strip port connector and the distal end of the test meter housing; and
employing the test meter to determine an analyte in a bodily fluid sample using an analytical test strip received in the strip port connector.

15. The method of claim 14 wherein the distal portion of the strip port connector includes the analytical test strip guide; and
wherein the strip port connector further includes a proximal portion disposed within the test meter housing, the proximal portion having at least one electrical connector pin configured for operable electrical contact with an analytical test strip received within the strip port opening, and
wherein the upper guide surface of the analytical test strip guide is configured such that the analytical test strip received in the strip port opening is guided and supported by the at least one upper guide surface.

16. The method of claim 15 wherein the analytical test strip guide includes at least one guide rail with the at least one upper guide surface, and
wherein the at least one liquid entrapment feature is a gap adjacent the guide rail, the gap configured to direct free liquid that enters the strip port opening away from the upper guide surface of the guide rail and to entrap such directed liquid in at least one of the distal portion of the strip port connector and the distal end of the test meter housing.

17. The method of claim 15 wherein the analytical test strip guide further includes at least one hemispherical protrusion configured to facilitate the directing of the free liquid.

18. The method of claim 17 wherein the at least one hemispherical protrusion is disposed on the at least one guide rail.

19. The method of claim 16 wherein the analytical test strip guide further includes at least one sidewall.

20. The method of claim 19 wherein the gap is between the guide rail and the sidewall.
